(19) United States
(12) Patent Application Publication
(10) Pub. No.: US 2026/0110688 A1
(43) Pub. Date: Apr. 23, 2026

(54) DETECTION OF CANCER BIOMARKERS USING NANOPARTICLES

(71) Applicant: THE UNIVERSITY OF MANCHESTER, Manchester (GB)

(72) Inventors: Kostas KOSTARELOS, Manchester (GB); Marilena HADJIDEMETRIOU, Strovolos (CY)

(73) Assignee: THE UNIVERSITY OF MANCHESTER, Manchester (GB)

(21) Appl. No.: 19/366,779

(22) Filed: Oct. 23, 2025

Related U.S. Application Data
(63) Continuation of application No. 18/152,843, filed on Jan. 11, 2023, now abandoned.

(51) Int. Cl. G01N 33/575; G01N 33/543

(52) U.S. Cl. CPC G01N 33/57585; G01N 33/5432; G01N 33/54346

(57) ABSTRACT

The present invention relates to methods for identifying and detecting potential disease specific biomarkers from biofluids. In particular, the methods involve in vivo administration of nanoparticles to a subject in a diseased state or incubating nanoparticles in a biofluid sample taken from a subject in a diseased state and analysis of the biomolecule corona formed on said nanoparticles. In addition, the present invention also relates to methods that provide a means to distinguish between a healthy and diseased state in a subject, such as for example the presence of a tumor in a human subject.

### CROSS-REFERENCE TO RELATED APPLICATIONS

This application claims priority to and is a continuation of U.S. patent application Ser. No. 18/152,843, filed on Jan. 11, 2023, which claims priority to and it is a continuation of U.S. patent application Ser. No. 16/330,358, filed on Mar. 4, 2019, which is is a national stage application (under 35 U.S.C. § 371) of PCT/EP2017/072349, filed Sep. 6, 2017, which claims benefit of United Kingdom Application No. 1615128.4, filed Sep. 6, 2016, which are incorporated herein by reference in their entireties.

### FIELD OF THE INVENTION

The present invention relates to methods for identifying and detecting potential disease specific biomarkers from biofluids. In particular, the methods involve administration of nanoparticles to a subject in a diseased state or incubation of nanoparticles in a biofluid sample taken from a subject in a diseased state and subsequent analysis of the biomolecule corona formed on said nanoparticles. In addition, the present invention also relates to methods that provide a means to distinguish between healthy and diseased states in a subject, such as for example the early detection of a tumor in a human subject or the presence or monitoring of the growth and/or response to treatment of a tumor in a human subject.

### BACKGROUND OF THE INVENTION

A biomarker, or biological marker, generally refers to a qualitative and/or quantitative measurable indicator of some biological state or condition. Biomarkers are typically molecules, biological species or biological events that can be used for the detection, diagnosis, prognosis and prediction of therapeutic response of diseases. Most biomarker research has been focused on measuring a concentration change in a known/suspected biomarker in a biological sample associated with a disease. Such biomarkers can exist at extremely low concentrations, for example in early stage cancer, and accurate determination of such low concentration biomarkers has remained a significant challenge.

Research into nanoparticle-based technologies for biomarker detection has been carried out but so far has failed to provide suitable methods to accurately identify/discover and detect biomarkers. One particular problem is that currently available laboratory tests detect only a minute fraction of potential biomarkers, due to their extremely low concentration in biofluids. In addition to the ‘swamping’ effect, caused by “non-specific” high abundant molecules, this causes significant difficulties. Indeed, the issue of “signal-to-noise” exceeds the current capability of proteomic analysis and therefore limits the diagnostic information that can be obtained.

Furthermore, such methods are mainly used to detect already known disease-specific molecules. For example, nanoparticle-based immunoassays, such as sandwich-type assays have been developed, where nanoparticles are used to fluorophore-label the secondary antibody to a known disease-specific molecule. The presence of a target biomarker generates a fluorescent signal that is detected using microscopy or a fluorescence spectrophotometer. NanoDLSay™ is a particular nanoparticle-based immunoassay that detects specific known biomarkers by measuring the size change of gold nanoparticle probes upon binding with target molecules. Gold nanoparticles conjugated to specific antibodies are mixed in vitro with biofluids. The binding of proteins with the gold nanoparticle immunoprobes can lead to nanoparticle cluster or aggregate formation. The average particle size increase of the assay solution is measured and correlated to the target protein concentration.

Nanoparticle-based immunoassays are inherently limited by the detection of a single molecule-biomarker. In order to increase the sensitivity and specificity of detection, biomarkers can, for example, be regarded as a panel of up- and down-regulated proteins, which differ in diseased and normal state. ‘Multiplexing’ is a difficult and highly expensive challenge with such technologies.

In vitro nanoparticle-based scavenger technologies such as Nanotrap® particle technology uses hydrogel nanoparticles with a porous outer shell that blocks the entry of high molecular weight proteins and an internal core which contains chemical affinity baits for interaction with low molecular weight proteins, after the introduction of nanoparticles into biological fluids. The limitation of this approach is that some of the high molecular weight proteins removed are known to act as ‘carriers’ for other proteins, possibly with biomarker potential.

Surprisingly, the inventors have found that analyzing the biomolecule corona formed on nanoparticles after following methods involving administration of nanoparticles to a subject in a diseased state or incubation of nanoparticles in a biofluid sample taken from a subject in a diseased state results in interaction of the nanoparticles with many more different types of biomolecules in comparison with analysis of biomolecules directly in biofluid samples. In one embodiment, the novel methods take advantage of the interaction of nanoparticles with biomolecules as a way to more comprehensively analyse the circulatory proteome and facilitate the detection of previously unknown disease-specific biomolecules.

In addition, inventors have also surprisingly found that particular methods of the invention can be employed to distinguish between healthy and disease states in a subject, for example the detection of the presence of a tumor or monitoring the growth and/or response to treatment of a tumor.

### SUMMARY OF THE INVENTION

In one aspect, the present invention relates to a method of identifying a biomarker from a biofluid, wherein the method comprises:
a. administering a plurality of nanoparticles to a subject in a diseased state to allow a biomolecule corona to form on the surface of said nanoparticles or incubating a plurality of nanoparticles in a biofluid sample taken from a subject in a diseased state to allow a biomolecule corona to form on the surface of said nanoparticles;
b. isolating the nanoparticles and surface-bound biomolecule corona; and
c. analyzing the biomolecule corona to identify the said biomarker.

The methods result in an interaction between the nanoparticles and a greater number of different types of proteins than can be detected by direct analysis of biofluids taken from a subject in a diseased state. It is to be understood that the method involves identification of a biomarker that provides a measurable indicator of some biological state or condition. This includes, but is not limited to, the discovery of unique disease-specific biomolecules (those biomolecules that are only present in a diseased state) but also includes detection of changes in biomolecule(s) that are present in both healthy and diseased states, for example upregulation or down regulation of biomolecules in a diseased state when compared to the healthy state. It will be understood that in order to identify a potential disease-specific biomarker, comparison against a suitable non-diseased control reference can be required. In one particular embodiment, the methods involve identifying panels of biomarkers (multiplexing), which can lead to increased sensitivity and specificity of detection. In a further particular embodiment, the methods facilitate the detection of previously unknown unique disease-specific biomolecules. In yet a further particular embodiment, the methods allow identification or detection of a biomarker without the need for invasive tissue sampling, e.g. a biopsy.

The methods are applicable to a wide range of nanoparticles and allow the benefit of removal of unbound and highly abundant biomolecules to allow identification of low abundant biomarkers that would otherwise be undetected. In addition to identification of potential biomarkers, the methods can also be employed to monitor changes in biomarkers, for example in response to therapy and/or to assist in diagnosis.

In another aspect, the present invention relates to a method of detecting a diseased state in a subject, wherein the method comprises:
a. administering a plurality of nanoparticles to a subject to allow a biomolecule corona to form on the surface of the nanoparticles;
b. isolating the nanoparticles and surface-bound biomolecule corona; and
c. determining the total biomolecule content of the biomolecule corona, which is determinative of the presence of disease in said subject.

Surprisingly, inventors have found that particular methods can be employed to distinguish between healthy and diseased states in a subject. Such methods can, for example, be useful in the early detection of a diseased state such as the presence of a tumor in a human subject.

The methods disclosed herein are applicable to any disease state in which identification and/or monitoring of biomarkers would be beneficial. Furthermore, particular methods of the invention, which an be employed to distinguish between healthy and diseased states in a subject, are applicable to a wide range of diseases, including but not limited to, cancer and neurodegenerative diseases.

### BRIEF DESCRIPTION OF DRAWINGS

FIG. 1 shows the SDS-Page gel for protein recovered directly from plasma compared with the analysis conducted following either in vivo administration of nanoparticles in mice or after in vitro incubation with plasma taken from mice. Data is presented for healthy or tumor bearing mice (i.e. lung adenocarcinoma model);

FIG. 2 shows the SDS-Page gel for protein recovered directly from plasma compared with the analysis conducted following either in vivo administration of nanoparticles in mice or after in vitro incubation with plasma taken from mice. Data is presented for healthy or tumor bearing mice (i.e. melanoma model);

FIG. 3 shows the number of proteins identified following in vivo administration of nanoparticles in mice or after in vitro incubation when compared with plasma. The data demonstrates that a significantly higher number of proteins were identified when compared to plasma. Data generated using lung adenocarcinoma model;

FIG. 4(a) provides a breakdown of the host proteins identified following in vivo administration;

FIG. 4(b) provides after in vitro incubation in the lung adenocarcinoma model, ranked by fold change in amount of that protein detected in the protein corona from healthy and diseased mice (proteins with greater than 5 fold change shown);

FIG. 5(a) provides a breakdown of the proteins identified following in vivo administration;

FIG. 5(b)A-5(b)D provides after in vitro incubation in the melanoma model ranked by fold change in amount of that protein detected in samples from healthy and diseased mice (proteins with greater than 5 fold change shown);

FIG. 6 provides data from a BCA assay for the lung adenocarcinoma model. The level of total amount of protein detected in the protein corona from tumor-bearing animals following in vivo administration is significantly greater than after in vitro incubation. Furthermore, there is a clear distinction between tumor and healthy mice in regards to the level of total amount of protein detected in the protein corona following in vivo administration;

FIG. 7 provides data from a BCA assay for the melanoma model. The level of total amount of protein detected in the protein corona from tumor-bearing animals following in vivo administration is significantly greater than after in vitro incubation. Furthermore, there is a clear distinction between tumor and healthy mice in regards to the level of total amount of protein detected in the protein corona following in vivo administration;

FIG. 8 shows the SDS-Page gel for protein recovered directly from plasma before and after infusion with Calex and compares this with analysis conducted following either in vivo administration of nanoparticles or after in vitro incubation of nanoparticles with plasma taken from the human patients and shows that the masking problem with protein recovered directly from plasma is not present;

FIG. 9 provides a breakdown of the top 10 proteins identified in the nanoparticle protein corona based on Relative Protein Abundance (represents the percentage of each protein relative to the total amount of protein purified from the protein corona of the nanoparticles) following in vivo administration to 6 patients. FIG. 9 also provides a list of the top 10 most abundant proteins found in plasma taken from one of the patients (Patient 1). The data demonstrates that the methods of the invention are able to extract out different types of proteins from the patients that are not readily detectable in plasma;

FIG. 10A-10C provides a breakdown of the top 200 proteins identified in the nanoparticle protein corona based on the RPA % following in vivo administration to 6 patients (RPA % data is based on the mean of the six patients). The data further demonstrates that the methods of the invention are able to extract out different types of proteins from the patients that are not readily detectable in plasma;

FIG. 11 provides the results for the BCA assay for the human patient study. The level of total amount of protein detected in the protein corona from patients following in vivo administration is significantly greater than after in vitro incubation of the nanoparticles in plasma taken from such patients;

FIG. 12 shows data before and after dosing of Dynamic light scattering (DLS), ζ-potential measurements and negative stain transmission electron microscopy (TEM) showing physicochemical characteristics of the PEGylated doxorubicin-encapsulated liposomes (Caelyx®);

FIG. 13 shows surface-bound proteins that were also classified according to their molecular mass;

FIG. 14 is a melanoma model study showing surface-bound proteins that were also classified according to their molecular mass; and

FIG. 15 is for a lung carcinoma study showing surface-bound proteins that were classified according to their molecular mass. A heatmap of RPA (%) of proteins identified in the coronas formed onto liposomes intravenously infused in health and lung-carcinoma mice, as identified by LC-MS/MS.

### METHOD FOR IDENTIFYING NEW BIOMARKERS

Methods provided by the present invention include those described generally above, and are further illustrated by all of the particular method steps disclosed herein.

The present invention relates to a method of identifying a biomarker from a biofluid, wherein the method comprises:
a. administering a plurality of nanoparticles to a subject in a diseased state to allow a biomolecule corona to form on the surface of said nanoparticles or incubating a plurality of nanoparticles in a biofluid sample taken from a subject in a diseased state to allow a biomolecule corona to form on the surface of said nanoparticles;
b. isolating the nanoparticles and surface-bound biomolecule corona; and
c. analyzing the biomolecule corona to identify the said biomarker.

The present invention relates to a method of identifying a biomarker from a biofluid.

In a particular embodiment of the present invention, the biomarker comprises at least one biomolecule (for example, a protein, peptide, fatty acid, lipid, amino acid, sugar, amide or nucleic acid) and the biofluid is selected from plasma, urine, saliva, lacrimal, cerebrospinal and ocular fluids. In a particular embodiment, the biofluid is plasma and the biomarker is at least one protein.

The present invention involves the use of a plurality of nanoparticles. The methods are applicable to any types of nanoparticles capable of attracting a biomolecule corona. In a particular embodiment, the nanoparticles are selected from liposomes, gold nanoparticles, polymeric nanoparticles, carbon nanotubes and graphene oxide nanoparticles.

Conveniently, the nanoparticles are liposomes. Liposomes are generally spherical vesicles comprising at least one lipid bilayer. Liposomes are often composed of phospholipids. In a particular embodiment, the liposomes are composed of phospholipid molecules and functionalised amphiphilic molecules (eg. PEGylated DSPE) that are able to self-assemble into unilamellar vesicles. Conveniently, the liposomes are able to encapsulate drug molecules in their inner aqueous phase.

The corona formed on the nanoparticles is a biomolecule corona. The term “biomolecule” in this context includes, but is not limited to, proteins, peptides, fatty acids, lipids, amino acids, amides, sugars and nucleic acids (such as for example different types of DNA or RNA). Conveniently, the biomolecule corona comprises a protein corona.

Step (a) of the method involves administering a plurality of nanoparticles to a subject in a diseased state to allow a biomolecule corona to form on the surface of said nanoparticles or incubating a plurality of nanoparticles in a biofluid sample taken from a subject in a diseased state to allow a biomolecule corona to form on the surface of said nanoparticles. Where the plurality of nanoparticles are administered to a subject in a diseased state to allow a biomolecule corona to form on the surface of said nanoparticles, administration can be by any route that allows the biomolecule corona to form. Suitable routes of administration include but are not limited to intravenous, oral, intracerebral (including spinal), intraperitoneal and intra-ocular. Conveniently, the route of administration is by intravenous injection. The biomolecule corona typically forms within less than 10 minutes from administration.

In the case where the plurality of nanoparticles are incubated in a biofluid sample taken from a subject in a diseased state, such incubation can be carried out in-vitro. Conveniently, this involves incubating at 37° C. the nanoparticles with plasma taken from the subject in the diseased state for a minimum period of 10 minutes, although it is possible that biomolecule corona can form immediately upon incubation. Conveniently, the mixture can be subject to agitation, for example by way of an orbital shaker set at approximately 250 rpm to mimic in vivo conditions.

Once the biomolecule corona has formed on said nanoparticles, said particles are isolated. Any isolation technique that is capable of preserving the surface-bound biomolecule corona is suitable. Conveniently, the nanoparticles with surface-bound biomolecule corona are isolated from the biofluid and purified to remove unbound and highly abundant biomolecules (for example albumin and/or immunoglobulins, which can constitute 90% of the plasma proteome) to allow identification of lower abundant biomarkers. The method therefore allows minimization of any masking caused by the highly abundant proteins. Conveniently, the isolation is achieved by a method comprising size exclusion chromatography followed by ultrafiltration. The method offers particular benefits over centrifugation methodologies that favour interaction with proteins. In a particular embodiment, the method allows identification of low molecular weight protein biomarkers. Conveniently, the method allows identification of protein biomarkers with molecular weight of less than 80 kDa. More conveniently, the method allows identification of protein biomarkers with molecular weight of less than 40 kDa or less than 20 kDa. In a particular embodiment of the invention, the beneficial sensitivity and high level of precision provided by the method allows the identification of intracellular protein disease related biomarkers that are present in low abundance and would otherwise be very difficult to identify.

Analysis of the biomolecule corona in order to identify biomarkers can be carried out using any suitable technique capable of detecting said biomarkers. In a particular embodiment of the invention, the biomolecule corona is analysed by gel electrophoresis, mass spectrometry, an immunoassay, UV-Vis. absorption, fluorescence spectroscopy, chromatography or NMR methodology. Conveniently, the biomolecule corona is analysed by mass spectrometry, which can allow qualitative and/or quantitative analysis of the biomolecule corona present on the nanoparticles. In a particular embodiment, certain methods may allow identification of unique biomolecules without the need for highly specialized and ultra-sensitive analytical mass spectrometry instrumentation such as an UltiMate® 3000 Rapid Separation LC (RSLC, Dionex Corporation, Sunnyvale, CA) coupled to a LTQ Velos Pro (Thermo Fisher Scientific, Waltham, MA) mass spectrometer. In one aspect of this embodiment, analysis of the biomolecule corona is carried out after administering a plurality of nanoparticles to a subject in a diseased state to allow a biomolecule corona to form on the surface of said nanoparticles and isolating the nanoparticles and surface-bound biomolecule corona. When compared to other methods, such methods can yield high levels of unique low abundant biomolecules and allow identification of such unique biomolecules without the need for highly specialized and ultra-sensitive analytical mass spectrometry instrumentation such as an UltiMate® 3000 Rapid Separation LC (RSLC, Dionex Corporation, Sunnyvale, CA) coupled to a LTQ Velos Pro (Thermo Fisher Scientific, Waltham, MA) mass spectrometer.

In addition to the identification of a single biomarker, the methods also provide the ability to identify panels of biomarkers (multiplexing). This approach can lead to increased sensitivity and specificity of detection.

In addition to the identification of new biomarkers, the methods also provide the ability to monitor changes in biomarkers for example in response to therapy. In one particular embodiment the therapy administered to the subject prior to testing is a drug molecule, such as for example, an anti-cancer compound. Suitable anti-cancer compounds include, but are not limited to, compounds with activity in cancers such as lung cancer, melanoma or ovarian cancer. Conveniently, the anti-cancer compound is doxorubicin.

In a particular embodiment, the invention relates to a method of identifying a biomarker from a biofluid, wherein the method comprises:
a. isolating a plurality of nanoparticles with surface-bound biomolecule corona from a biofluid sample taken from a subject in a diseased state; and
b. analyzing the biomolecule corona to identify the said biomarker.

### Method of Detecting a Diseased State in a Subject

The present invention also relates to a method of detecting a diseased state in a subject, wherein the method comprises:
a. administering a plurality of nanoparticles to a subject to allow a biomolecule corona to form on the surface of the nanoparticles;
b. isolating the nanoparticles and surface-bound biomolecule corona; and
c. determining the total biomolecule content of the biomolecule corona, which is determinative of the presence of disease in said subject.

Surprisingly, inventors have found that the analysis of the total biomolecule content of the biomolecule corona isolated after administering a plurality of nanoparticles to a subject to allow a biomolecule corona to form on the surface of the nanoparticles is indicative of the presence of disease in said subject. In a particular embodiment, the biomolecule is a protein.

The total biomolecule content of the biomolecule corona can be determined by any method capable of quantifying the level of said biomolecules in the surface-bound corona. In one embodiment, the biomolecule method involves determining the total protein content and this is suitably determined by a bicinchoninic acid assay (BCA assay). In one particular embodiment, the subject is a human patient and the total protein content is at least 700, 800, 900, 1000, 1250, 1500, 1800, 2000, 25000 or 3000Pb when measured using a BCA assay. In yet a further aspect of this embodiment, the disease state is cancer, such as for example ovarian cancer, lung cancer or melanoma.

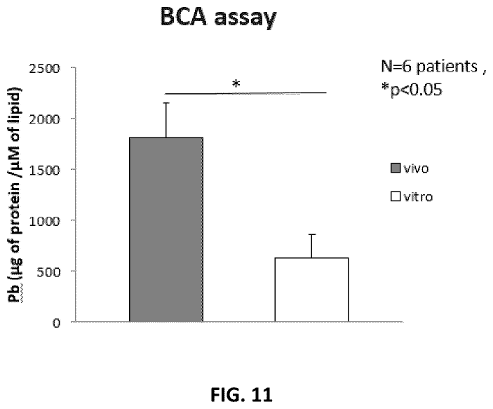

Surprisingly, inventors have also found that the total protein content determined by the inventive method is greater than if determined by incubating the plurality of nanoparticles in-vitro with a biofluid taken from the subject. FIG. 11 shows data to illustrate this surprising discovery. In a particular embodiment, the total protein content determined is at least between 1.2 and 5 fold higher than if determined by incubating the plurality of nanoparticles in-vitro with a biofluid isolated from the subject. Conveniently, total protein content determined is at least 1.5, 1.8, 2, 3, 4 or 5 fold higher than if determined by incubating the plurality of nanoparticles in-vitro with a biofluid isolated from the subject. Conveniently, the subject in this embodiment is a human.

Conveniently, the route of administration of the nanoparticles is by intravenous injection. The biomolecule corona typically forms within less than a few minutes from administration.

Any isolation technique that is capable of preserving the surface-bound biomolecule corona is suitable. Conveniently, the nanoparticles with surface-bound biomolecule corona are isolated from the biofluid and purified to remove unbound and highly abundant biomolecules (for example albumin) to allow identification of lower abundant biomarkers. The method therefore allows minimization of any masking caused by the highly abundant proteins. Conveniently, the isolation is achieved by a method comprising size exclusion chromatography followed by ultrafiltration.

In addition to a determination of the total biomolecule content of the biomolecule corona, analysis of the biomolecule corona can also reveal qualitative and quantitative information regarding specific potential biomarkers. Such analysis can be carried out using any suitable techniques of capable of detecting said biomarkers. In a particular embodiment of the invention, the biomolecule corona is analysed by mass spectrometry, an immunoassay, UV-Vis. absorption, fluorescence spectroscopy, chromatography or NMR methodology. Conveniently, the biomolecule corona is analysed by mass spectrometry, which can allow qualitative and quantitative analysis of the biomolecule corona present on the nanoparticles. In a particular embodiment, the methods allow identification of unique biomolecules without the need for highly specialized and ultra-sensitive analytical mass spectrometry instrumentation such as using an UltiMate® 3000 Rapid Separation LC (RSLC, Dionex Corporation, Sunnyvale, CA) coupled to a LTQ Velos Pro (Thermo Fisher Scientific, Waltham, MA) mass spectrometer.

A further aspect of the invention includes novel biomarkers that are linked with particular diseases, such as ovarian cancer, lung cancer or melanoma and could facilitate personalized healthcare and patient selection and stratification strategies for therapy. Examples of such biomarkers are further described in the experimental examples below.

### EXAMPLES

Further examples of the invention are described hereinbelow, by way of example only, with reference to the accompanying figures.

### Example 1. Xenograft Mice Models

Six to eight week old male nude SCID beige mice were purchased from Charles River (UK). Five to six week old female C57BL/6 mice were purchased from Charles River (UK). Animal procedures were performed in compliance with the UK Home Office Code of Practice for the Housing and Care of Animals used in Scientific Procedures. Mice were housed in groups of five with free access to water and kept at temperature of 19-22° C. and relative humidity of 45-65%. Before performing the procedures, animals where acclimatized to the environment for at least 7 days.

### Lung Adenocarcinoma Model:

Six to eight weeks old male nude SCID beige mice were intravenously injected (via tail vein) with a549-luc cells (5E6 cell/200 ul of PBS).

### Melanoma Model:

Five to six week old female C57BL/6 mice were subcutaneously injected (to the left leg) with 0.5×10⁶ of B16F10-luc melanoma cells in a volume of 50 μl of PBS.

### Preparation of Liposome Nanoparticles

### Materials

Hydrogenated soy phosphatidylcholine (HSPC), 1,2-distearoyl-sn-glycero-3-phosphoethanolamine-N-[methoxy(polyethylene glycol)-2000 (DSPE-PEG2000) were purchased from Avanti Polar Lipids (USA), while cholesterol was purchased from Sigma (UK).

### Preparation Methods

Liposomes with a composition of (HSPC:CHOL:DSPE-PEG2000 (56.3:38.2:5.5) were prepared by thin lipid film hydration method followed by extrusion. Briefly, lipids were dissolved in chloroform:methanol mixture (4:1) in a total volume of 2 ml, using a 25 ml round bottom flask. Organic solvents were then evaporated using a rotary evaporator (Buchi, Switzerland) at 40° C., at 150 rotations/min, 1 h under vacuum. Lipid films were hydrated with ammonium sulphate 250 mM (pH 8.5) at 60° C. to produce large multilamellar liposomes. Small unilamellar liposomes were then produced by extrusion though 800 nm and 200 nm polycarbonate filters (Whatman, VWR, UK) 10 times each and then 15 times through 100 nm and 80 nm extrusion filters (Whatman, VWR, UK) using a mini-Extruder (Avanti Polar Lipids, Alabaster, AL).

Protein Corona Formation after In Vivo Administration of Nanoparticles in Mice

Mice were anesthetized by inhalation of isoflurane and liposomes were administered intravenously via the lateral tail vein, at a lipid dose of 0.125 mM/g body weight, used for preclinical studies. 10 minutes post-injection, blood was recovered by cardiac puncture using K2EDTA coated blood collection tubes. Plasma was prepared by inverting the collection tubes 10 times to ensure mixing of blood with EDTA and subsequent centrifugation for 12 minutes at 1300 RCF at 4° C. Supernatant was collected into Protein LoBind Eppendorf Tubes. The plasma samples obtained from three mice were pooled together.

Protein Corona Formation after In Vitro Incubation with Plasma Taken from Mice

The in vitro formed corona was allowed to form using a liposome concentration of 2.25 mM, which is equivalent to the typical concentration of liposomes found in plasma after in vivo administration to mice as described above. For all in vitro protein binding studies, 2.25 mM of liposomes (180 μl of 12.5 mM) were incubated with 820 μl of mouse plasma for 10 min at 37° C. in orbital shaker at 250 rpm setting to mimic in vivo conditions.

Separation of Corona-Coated Nanoparticles from Unbound and Weakly Bound Proteins

Nanoparticles recovered from the in vivo or in vitro experiments described above were separated from excess plasma proteins by size exclusion chromatography followed by membrane ultrafiltration. Immediately after in vitro or in vivo incubations, 1 ml of plasma samples was loaded onto a Sepharose CL-4B (SIGMA-ALDRICH) column (15×1.5 cm) equilibrated with HBS. Stewart assay in each chromatographic fraction (1 ml) revealed that nanoparticles were eluted in fractions 4, 5 and 6. Fractions containing nanoparticles were then pooled together and concentrated to 500 μl by centrifugation using Vivaspin 6 column (10000 MWCO, Sartorious, Fisher Scientific) at 9000 rpm. Vivaspin 500 centrifugal concentrator (1 000 000 MWCO, Sartorious, Fisher Scientific) was then used at 9000 rpm, to further concentrate the samples to 100 μl and to ensure separation of protein-coated nanoparticles from the remaining large unbound proteins. Nanoparticles were then washed 3 times with 100 μl HBS to remove weekly bound proteins.

### SDS-PAGE Electrophoresis

Samples of protein corona-coated nanoparticles were mixed with 20 ul of Tris-Glycine SDS buffer, with 4 ul of NuPAGE REDUCING and with water for a final volume of 40 μl and boiled for 5 minutes at 90° C. Samples were then loaded in 4-20% NOVEX Tris-Glycine Protein Gel (ThermoFisher Scientific). The gel was run for 25-40 minutes at 225V, until the proteins neared the end of the gel, in 10 times diluted Novex® Tris-Glycine SDS Running Buffer (ThermoFisher Scientific). Staining was performed with Imperial Gel Staining reagent (Sigma Life Science) for an hour followed by washing in distilled water for 2 days.

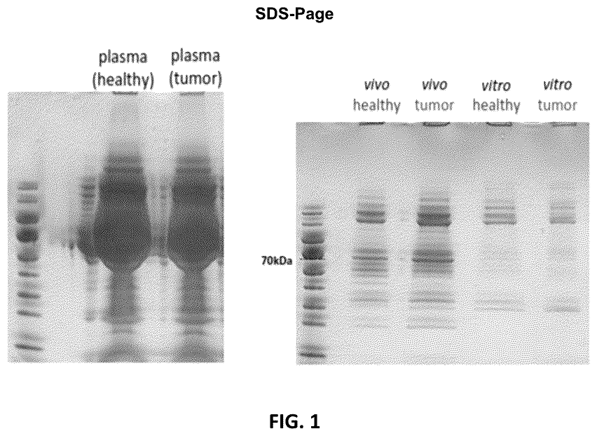

FIG. 1 shows the SDS-Page gel for protein recovered directly from plasma (healthy and tumor, i.e. lung adenocarcinoma model) where a large signal is present as a result of highly abundant proteins such as albumin. In contrast, the SDS-Page gel for analysis conducted following either in vivo administration of nanoparticles in mice or after in vitro incubation with plasma taken from mice, does not exhibit the masking problem and the data reveals the presence of a number of lower abundant proteins.

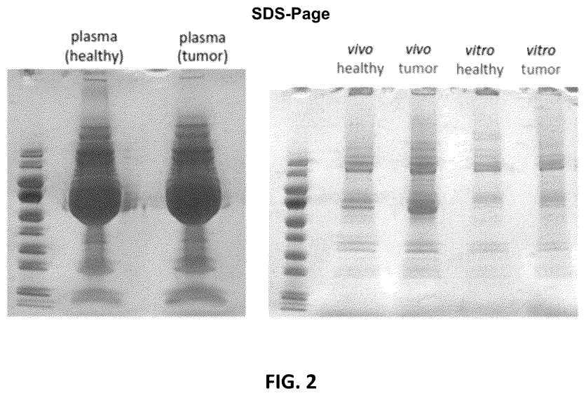

Similar differences are also evident for the melanoma model (FIG. 2).

### Mass Spectrometry

Bands of interest were excised from the SDS-Page gel and dehydrated using acetonitrile followed by vacuum centrifugation. Dried gel pieces were reduced with 10 mM dithiothreitol and alkylated with 55 mM iodoacetamide. Gel pieces were then washed alternately with 25 mM ammonium bicarbonate followed by acetonitrile. This was repeated, and the gel pieces dried by vacuum centrifugation. Samples were digested with trypsin overnight at 37° C.

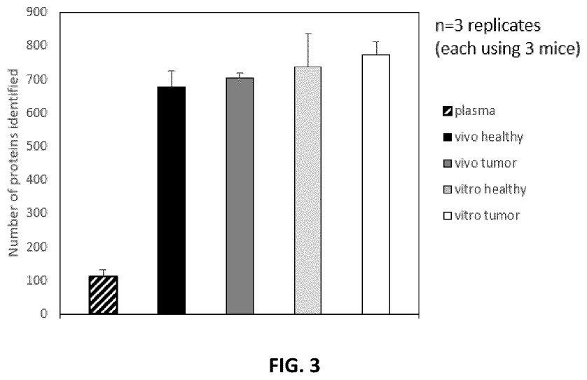

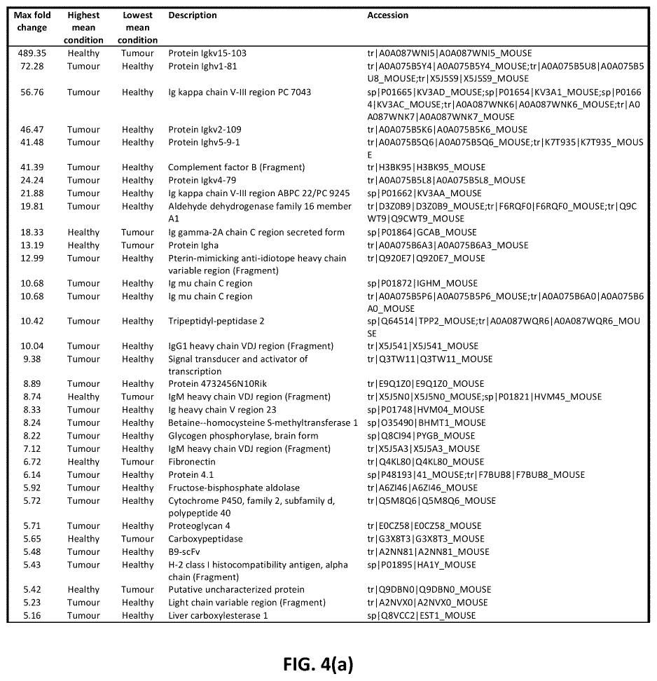

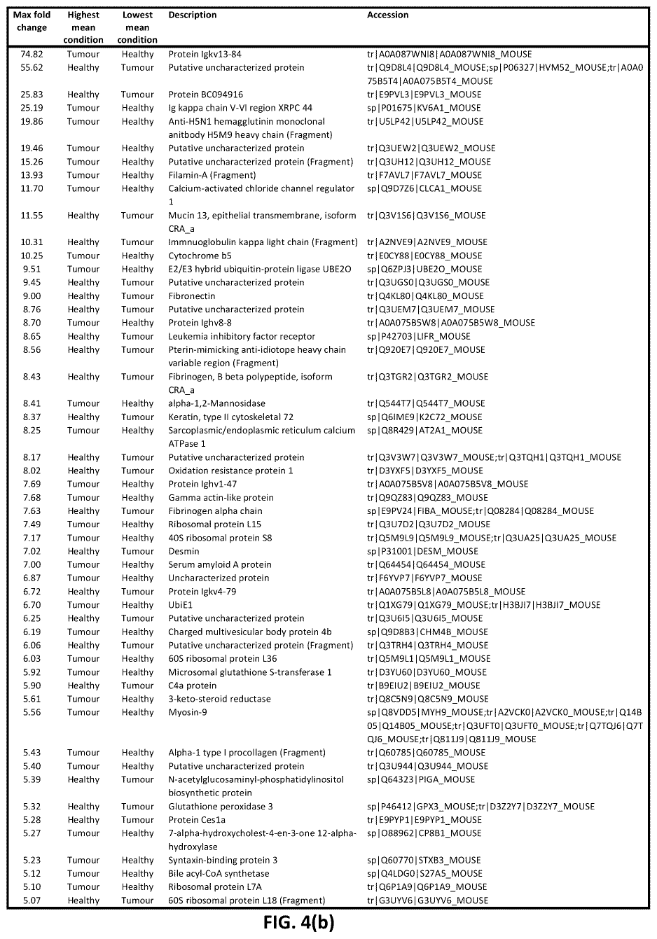

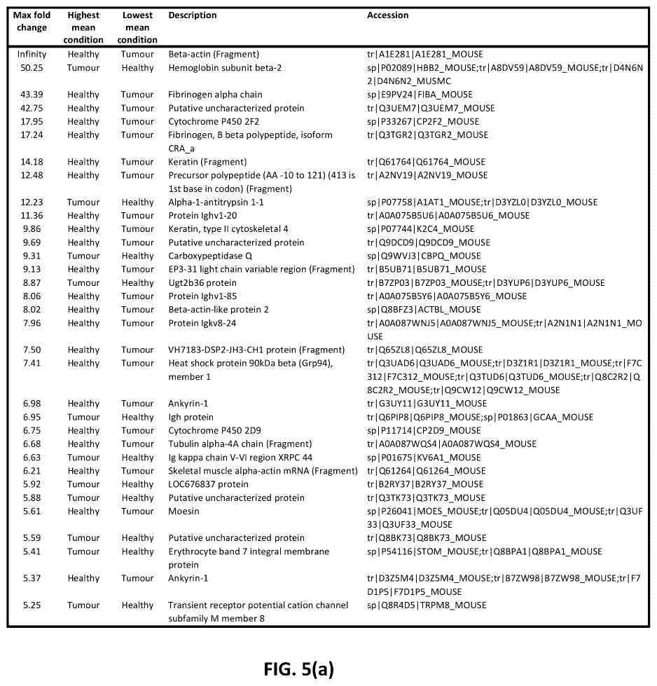

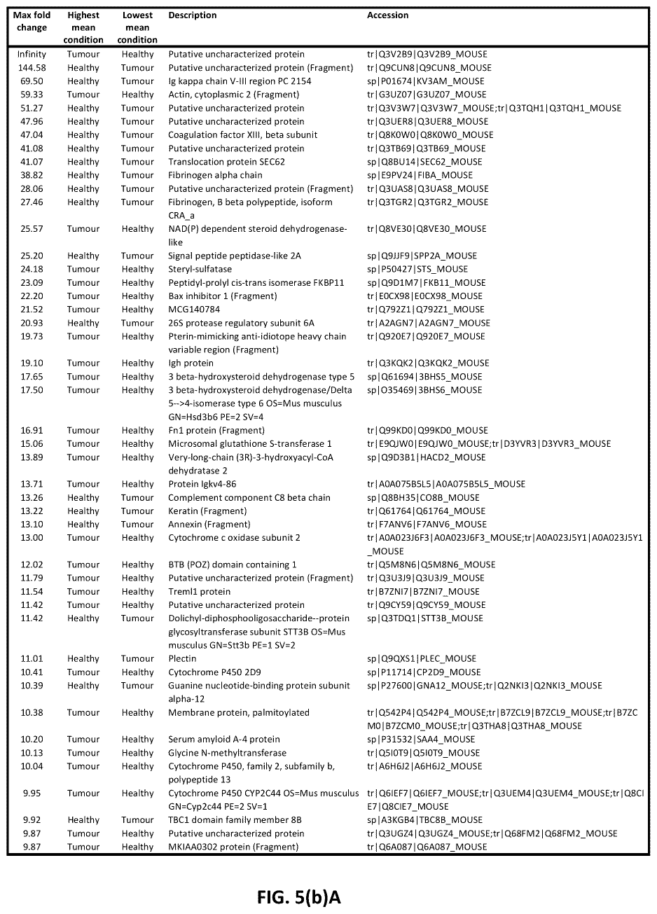

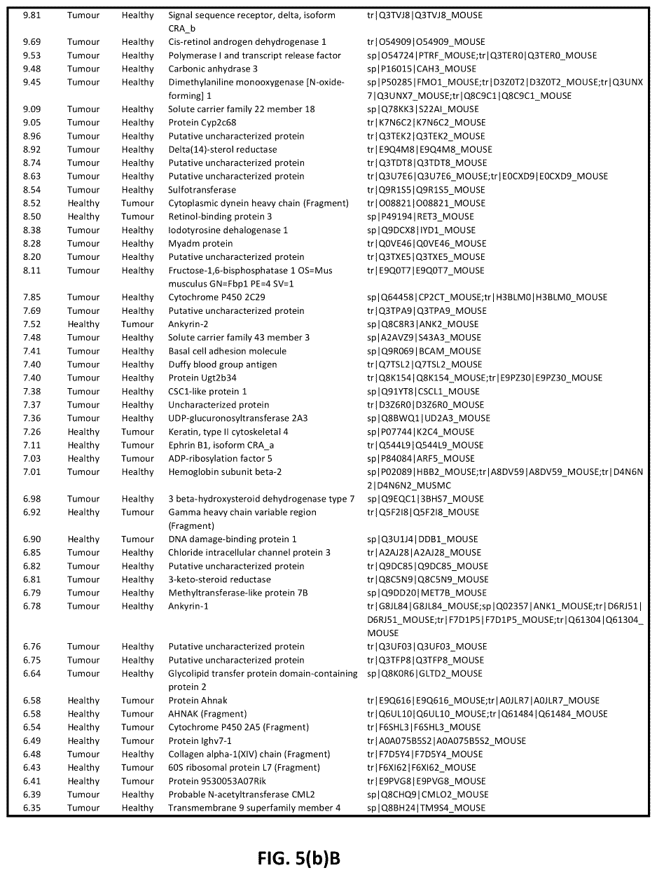

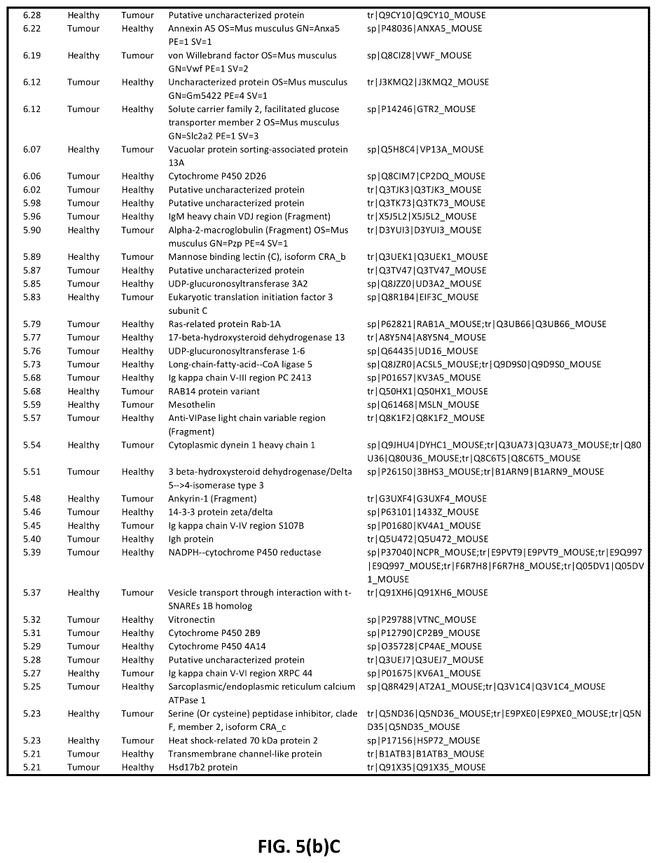

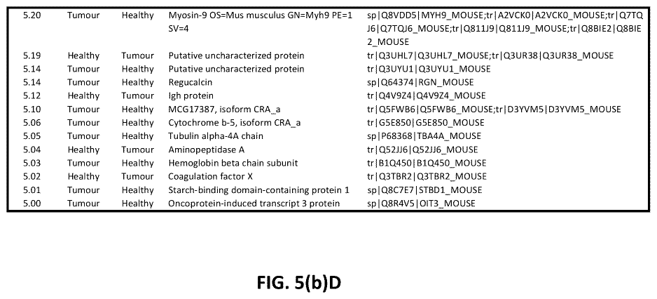

Digested samples were analysed by LC-MS/MS using an UltiMate® 3000 Rapid Separation LC (RSLC, Dionex Corporation, Sunnyvale, CA) coupled to Orbitrap Velos Pro (Thermo Fisher Scientific, Waltham, MA) mass spectrometer. Peptide mixtures were separated using a gradient from 92% A (0.1% FA in water) and 8% B (0.1% FA in acetonitrile) to 33% B, in 44 min at 300 nL min⁻¹, using a 250 mm×75 μm i.d. 1.7 μM BEH C18, analytical column (Waters). Peptides were selected for fragmentation automatically by data dependent analysis. Data produced were searched using Mascot (Matrix Science UK), against the uniprot_concat2014_201410.fasta database. Data presented in FIG. 3 was validated using Scaffold (Proteome Software, Portland, OR) and data presented in FIGS. 4a, 4b, 5a and 5b were validated using Progenesis QI for Proteomics.

The Scaffold software (version Scaffold_4.4.5, Proteome Software Inc., Portland, OR) was used to validate MS/MS based peptide and protein identifications and for relative quantification based on spectral counting. Peptide identifications were accepted if they could be established at greater than 50.0% probability by the Peptide Prophet algorithm with Scaffold delta-mass correction. Protein identifications were accepted if they could be established at greater than 99.0% probability and contained at least 2 identified peptides. Protein probabilities were assigned by the Protein Prophet algorithm. Proteins that contained similar peptides and could not be differentiated based on MS/MS analysis alone were grouped to satisfy the principles of parsimony.

The improved ability to identify a greater number of proteins is also demonstrated in FIG. 3 (lung adenocarcinoma model), which shows the number of proteins identified following in vivo administration of nanoparticles in mice or after in vitro incubation with mouse plasma when compared with plasma analysis (data presented is the result of analysis conducted with Scaffold software as described above). The data demonstrates that a significantly higher number of corona proteins identified when compared to plasma analysis.

FIG. 4(a) provides a breakdown of the host proteins identified following in vivo administration and FIG. 4(b) provides after in vitro incubation in the lung adenocarcinoma model, ranked by fold change in amount of that protein detected in the protein corona from healthy and diseased mice (proteins with greater than 5 fold change shown). The data demonstrates that the methods are able to identify changes in proteins between tumor and healthy groups. Particular human tumour specific proteins were also identified in the nanoparticle protein corona of diseased mice in this lung adenocarcinoma model that involves the use of the human derived a549-luc cancer cell line. This demonstrates the ability of the method to detect proteins secreted by the human tumour. Data presented is the result of analysis conducted with Progenesis QI for Proteomics as described above.

FIG. 5(a) provides a breakdown of the proteins identified following in vivo administration and FIG. 5(b) provides after in vitro incubation in the melanoma model ranked by fold change in amount of that protein detected in samples from healthy and diseased mice. The data demonstrates that the methods are able to identify changes in proteins between tumor and healthy groups. Data presented is the result of analysis conducted with Progenesis QI for Proteomics as described above.

The accession numbers of the proteins indicated in FIGS. 4a, 4b, 5a and 5b were assigned using uniprot_concat2014_201410.fasta database.

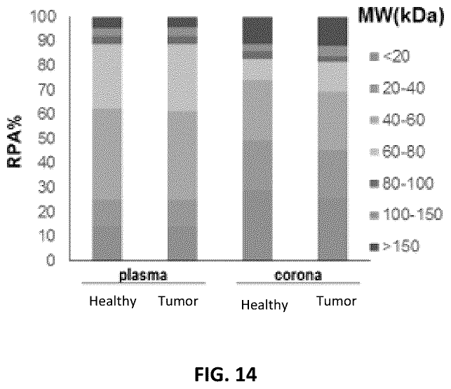

For the melanoma model study, surface-bound proteins were also classified according to their molecular mass as illustrated in FIG. 14. The RPA % values for each molecular weight group represents the average of 3 biological replicates (n=3 mice/replicate). As illustrated in FIG. 14, proteins with MW<60 kDa accounted for approximately 70% of the protein coronas formed, in both healthy and tumor-inoculated mice. Remarkably, analysis of the in vivo protein coronas increased the identification of proteins with MW<40 kDa, in comparison with plasma control analysis.

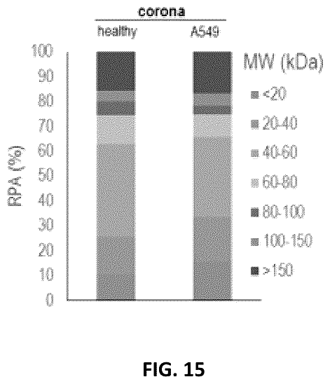

For the lung carcinoma study, surface-bound proteins were classified according to their molecular mass. A heatmap of RPA (%) of proteins identified in the coronas formed onto liposomes intravenously infused in healthy and lung-carcinoma mice, as identified by LC-MS/MS is shown in FIG. 15. Only proteins with RPA>0.5% are shown. RPA (%) values represent the average of 3 biological replicates (n=3 mice/replicate). Protein coronas formed in SCID mice were mainly composed of low MW proteins (>60% of corona proteins had a MW<60 kDa).

### Quantification of Adsorbed Proteins

Proteins associated with recovered nanoparticles were quantified by BCA Protein assay kit. Pb values, expressed as μg of protein/μM lipid were then calculated. For the BCA assay, a 6-point standard curve was generated by serial dilutions of BSA in HBS, with the top standard at a concentration of 2 μg/ml. BCA reagent A and B were mixed at a ratio of 50:1 and 200 μl of the BCA mixture were dispensed into a 96-well plate, in duplicates. Then, 25 μl of each standard or unknown sample were added per well. The plate was incubated for 30 minutes at 37° C., after which the absorbance was read at 574 nm on a plate reader (Fluostar Omega). Protein concentrations were calculated according to the standard curve. To quantify lipid concentration, 20 μl of each sample was mixed with 1 ml of chloroform and 500 μl of Stewart assay reagent in an Eppendorf tube. The samples were vortexed for 20 seconds followed by 1 min of centrifugation at 13 000 RPM. 200 μl of the chloroform phase was transferred to a quartz cuvette. The optical density was measured on a using Cary 50 Bio Spectrophotometer (Agilent Technologies) at 485 nm. Lipid concentration was calculated according to a standard curve.

Statistical analysis of the data was performed using IBM SPSS Statistics software. One-way analysis of variance (ANOVA) followed by the Tukey multiple comparison test were used and p values <0.05 were considered significant.

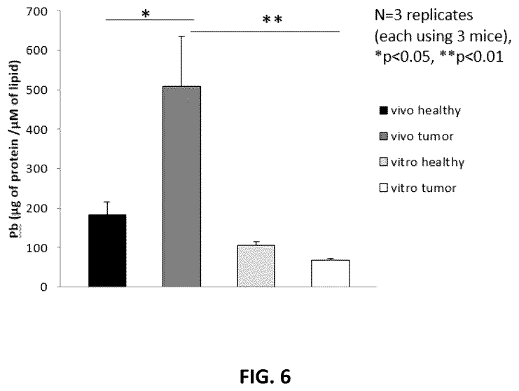

FIG. 6 provides the results for the BCA assay for the lung adenocarcinoma model. Surprisingly, the level of total amount of protein detected in tumor-bearing animals following in vivo administration is significantly greater than after in vitro incubation. Furthermore, there is a clear distinction between tumor and healthy mice in regards to the level of total amount of protein detected in the protein corona following in vivo administration.

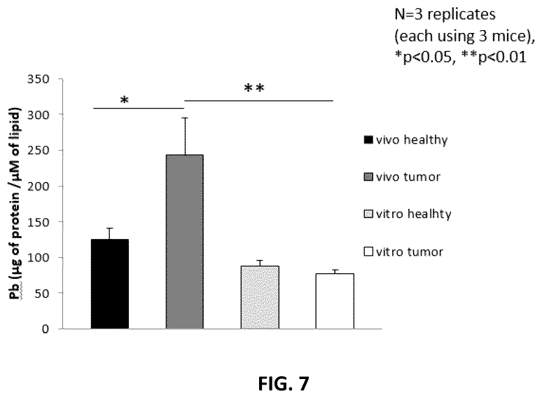

A similar effect is observed for the for the melanoma model (FIG. 7).

### Example 2 Human Experiments

### Subjects

Eligible patients included women with recurrent ovarian cancer commencing liposomal doxorubicin (Caelyx) treatment for the first time.

### Nanoparticles

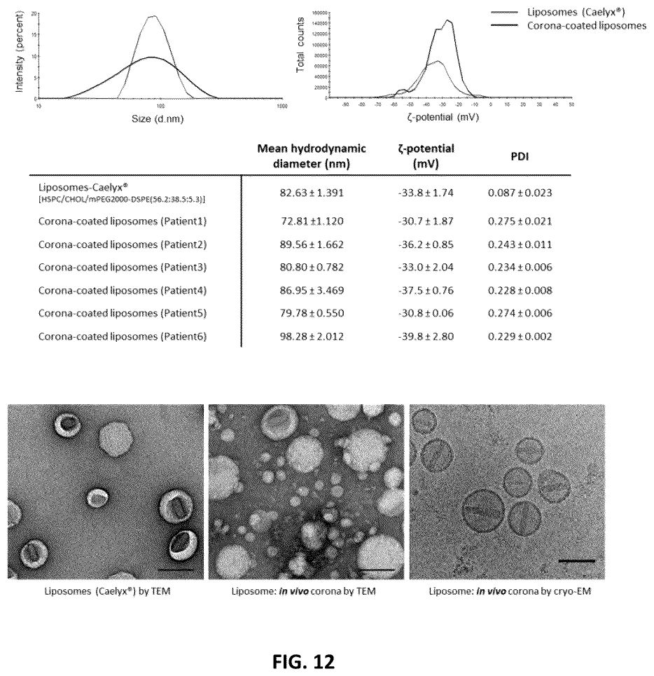

Dynamic light scattering (DLS), ζ-potential measurements and negative stain transmission electron microscopy (TEM) data showing physicochemical characteristics of the PEGylated doxorubicin-encapsulated liposomes (Caelyx®) employed in this study before and after dosing are summarised in FIG. 12.

### Dosing and Blood Sample Collection

Patients were intravenously infused with Caelyx (diluted in 5% dextrose) at a dose of 40 mg/m² for approximately 1.5 h. Collection of paired plasma samples (i.e. before and after cycle 1 infusion) were collected into commercially available anticoagulant-treated tubes (K2 EDTA BD Vacutainer). Plasma was then prepared by inverting the collection tubes 10 times to ensure mixing of blood with EDTA and subsequent centrifugation for 12 minutes at 1300 RCF at 4° C. Following centrifugation supernatant was immediately collected into labelled Protein LoBind Eppendorf Tubes and samples were maintained on ice while handling.

Protein Corona Formation after In Vitro Incubation of Nanoparticles with Plasma Taken from Patients Before Infusion

The in vitro formed corona was allowed to form using a liposome concentration of 0.3 mM in plasma taken from patients before infusion, which is equivalent to the concentration of liposomes found in 1 ml of recovered plasma after in vivo administration to humans as described above. For all in vitro protein binding studies, 0.3 mM of liposomes (20 μl of 17 mM) were incubated with 980 μl of human patient plasma for 10 min at 37° C. in orbital shaker at 250 rpm setting to mimic in vivo conditions.

Separation of Corona-Coated Nanoparticles from Unbound and Weakly Bound Proteins

Nanoparticles recovered from the in vivo human studies or the in vitro incubation method described above were separated from excess plasma proteins by size exclusion chromatography followed by membrane ultrafiltration. Immediately after in vitro and in vivo incubations, 1 ml of plasma samples was loaded onto a Sepharose CL-4B (SIGMA-ALDRICH) column (15×1.5 cm) equilibrated with HBS. Stewart assay in each chromatographic fraction (1 ml) revealed that nanoparticles were eluted in fractions 4, 5 and 6. Fractions containing nanoparticles were then pooled together and concentrated to 500 μl by centrifugation using Vivaspin 6 column (10000 MWCO, Sartorious, Fisher Scientific) at 9000 rpm. Vivaspin 500 centrifugal concentrator (1 000 000 MWCO, Sartorious, Fisher Scientific) was then used at 9000 rpm, to further concentrate the samples to 100 μl and to ensure separation of protein-coated nanoparticles from the remaining large unbound proteins. Nanoparticles were then washed 3 times with 100 μl HBS to remove weekly bound proteins.

### SDS-PAGE Electrophoresis

Samples of protein corona-coated nanoparticles were mixed with 20 μl of Tris-Glycine SDS buffer, with 4 ul of NuPAGE REDUCING and with water for a final volume of 40 μl and boiled for 5 minutes at 90° C. Samples were then loaded in 4-20% NOVEX Tris-Glycine Protein Gel (ThermoFisher Scientific). The gel was run for 25-40 minutes at 225V, until the proteins neared the end of the gel, in 10 times diluted Novex® Tris-Glycine SDS Running Buffer (ThermoFisher Scientific). Staining was performed with Imperial Gel Staining reagent (Sigma Life Science) for an hour followed by washing in distilled water for 2 days.

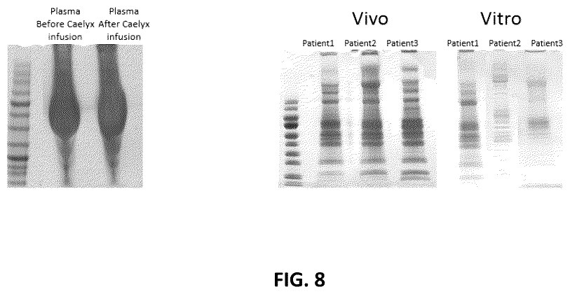

FIG. 8 shows the SDS-Page gel for protein recovered directly from plasma before and after infusion with Calex. A large signal is present as a result of highly abundant proteins such as albumin. In contrast, the SDS-Page gel for analysis conducted following either in vivo administration of nanoparticles or after in vitro incubation of nanoparticles with plasma taken from the human patients shows that the masking problem is not present. The data shows the presence of a number of lower abundant proteins that would not be detectable by analyzing the plasma directly. Evidently, the amount of this lower abundant protein that would not be detectable by analyzing the plasma directly is higher for the samples analysed after in vivo administration of nanoparticles than the samples analysed after in vitro incubation with plasma taken from the human patients. This benefit may further aid detection of potential unique biomolecules by reducing the need for particular highly specialized and ultra-sensitive analytical mass spectrometry instrumentation.

### Mass Spectrometry

Bands of interest were excised from the SDS-Page gel and dehydrated using acetonitrile followed by vacuum centrifugation. Dried gel pieces were reduced with 10 mM dithiothreitol

and alkylated with 55 mM iodoacetamide. Gel pieces were then washed alternately with 25 mM ammonium bicarbonate followed by acetonitrile. This was repeated, and the gel pieces dried by vacuum centrifugation. Samples were digested with trypsin overnight at 37° C.

Digested samples were analysed by LC-MS/MS using an UltiMate® 3000 Rapid Separation LC (RSLC, Dionex Corporation, Sunnyvale, CA) coupled to a Q Exactive™ Hybrid Quadrupole-Orbitrap™ (Thermo Fisher Scientific, Waltham, MA) mass spectrometer. Peptide mixtures were separated using a gradient from 92% A (0.1% FA in water) and 8% B (0.1% FA in acetonitrile) to 33% B, in 44 min at 300 nL min⁻¹, using a 250 mm×75 μm i.d. 1.7 μM BEH C18, analytical column (Waters). Peptides were selected for fragmentation automatically by data dependent analysis. Data produced were searched using Mascot (Matrix Science UK), against the SwissProt_2016_04 databasedatabase. Data were validated using Scaffold (Proteome Software, Portland, OR).

The Scaffold software (version Scaffold_4.4.5, Proteome Software Inc., Portland, OR) was used to validate MS/MS based peptide and protein identifications and for relative quantification based on spectral counting. Peptide identifications were accepted if they could be established at greater than 50.0% probability by the Peptide Prophet algorithm with Scaffold delta-mass correction. Protein identifications were accepted if they could be established at greater than 99.0% probability and contained at least 2 identified peptides. Protein probabilities were assigned by the Protein Prophet algorithm. Proteins that contained similar peptides and could not be differentiated based on MS/MS analysis alone were grouped to satisfy the principles of parsimony. Semi quantitative assessment of the protein amounts was conducted using normalized spectral countings, NSCs, provided by Scaffold Software. The mean value of NSCs obtained in the three experimental replicates for each protein was normalized to the protein MW and expressed as a relative quantity by applying the following equation:

$${MWNSC}_{k} = \frac{{( NSC / MW )}_{k}}{{\sum }_{i = 1}^{N} {( NSC / MW )}_{i}} \times 100 \qquad ( 1 )$$

where, MWNSCk is the percentage molecular weight normalized NSC for protein k and MW is the molecular weight in kDa for protein k. This equation takes into consideration the protein size and evaluates the contribution of each protein reflecting its relative protein abundance (RPA).

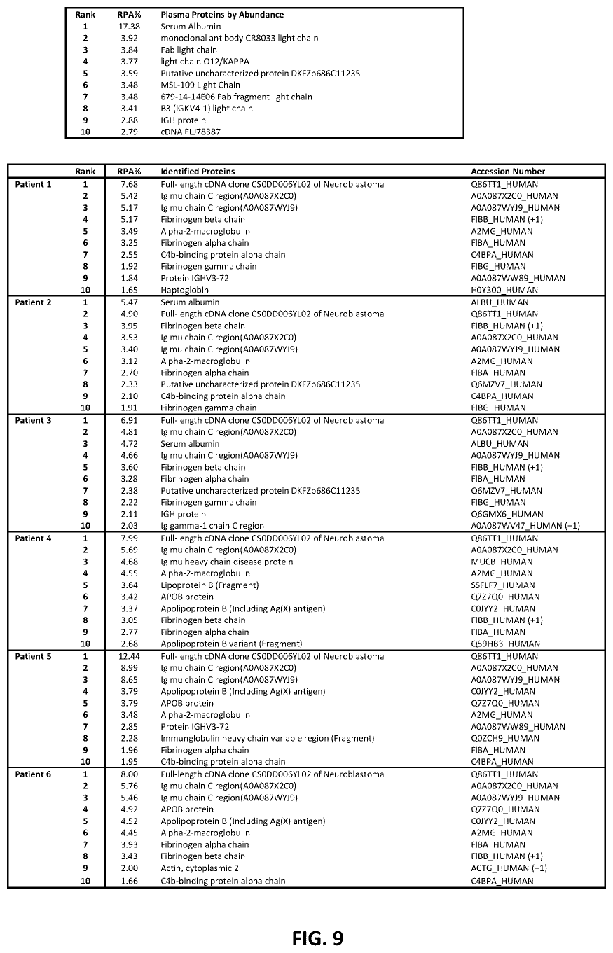

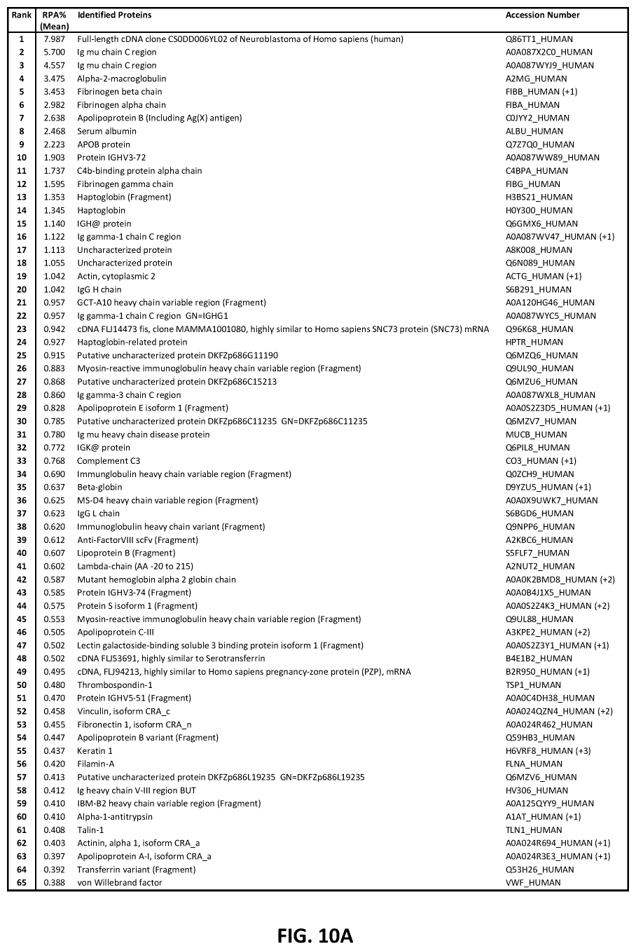

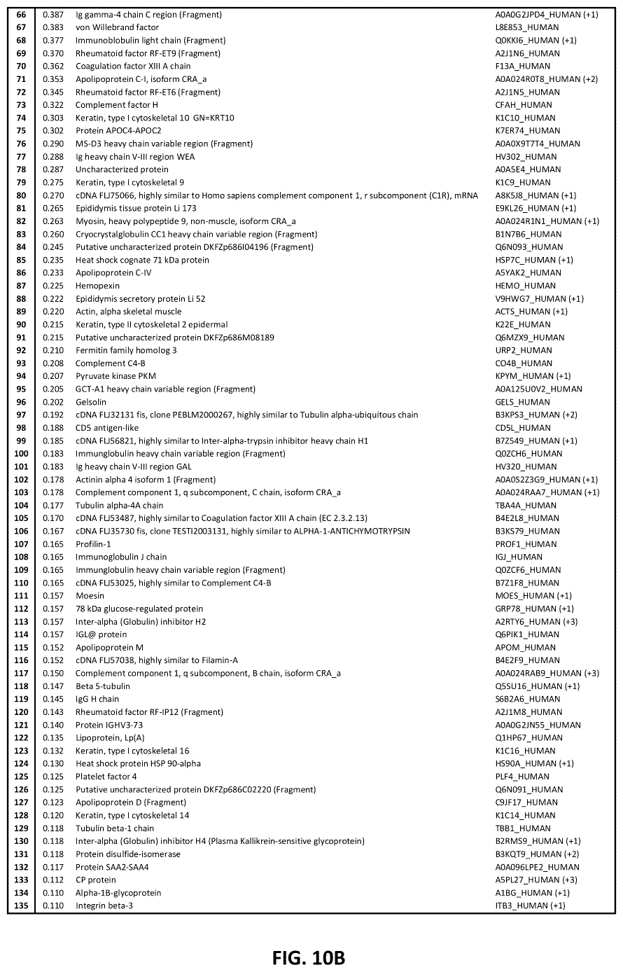

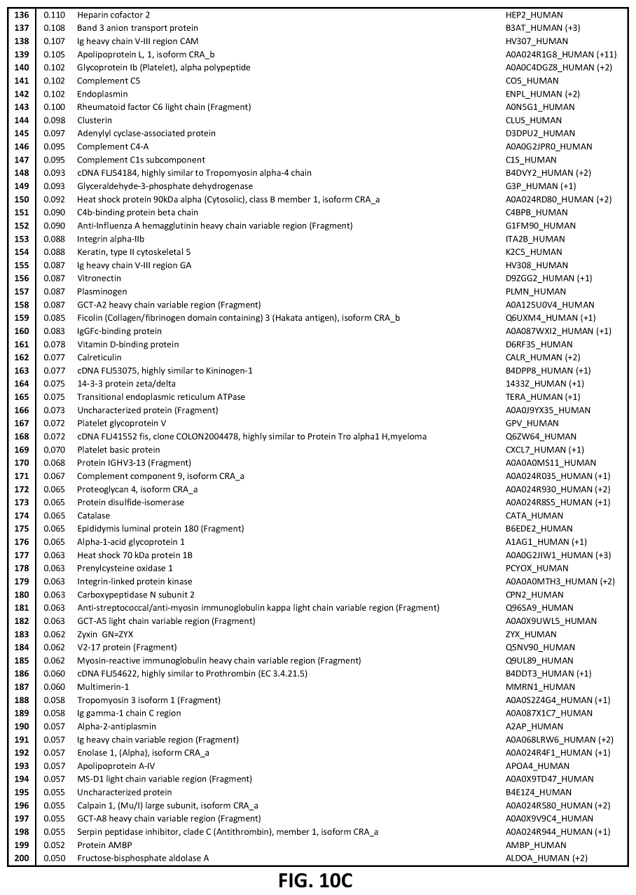

FIG. 9 provides a breakdown of the top 10 proteins identified with respect to percentage abundance following in vivo administration to 6 patients. The data demonstrates that the methods are able to extract out different types of proteins from the patients that are not readily detectable in plasma but are connected with cancer. FIG. 10 provides an extended list of proteins identified.

The accession numbers of the proteins indicated in FIG. 9 and FIG. 10 were assigned using SwissProt_2016_04 database.

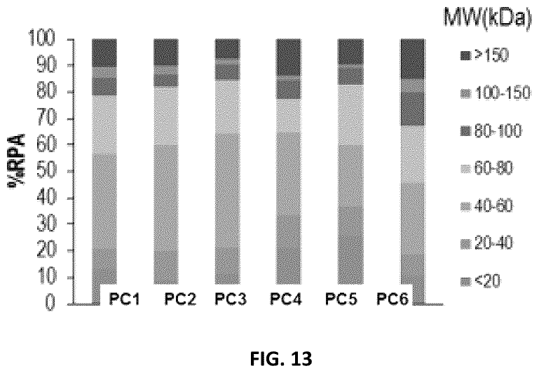

Surface-bound proteins were also classified according to their molecular mass as illustrated in FIG. 13. The percentage of relative protein abundance (% RPA) for each molecular weight group represent the average from 6 ovarian carcinoma patients. Plasma proteins with MW<80 kDA accounted for almost 80% of the protein coronas formed. It is possible that the low MW proteins identified (FIG. 13) have high affinity and interact directly with the surface of PEGylated liposomes and/or they are trapped between other corona-carrier proteins that are adhered to the nanoparticles surface.

### Quantification of Adsorbed Proteins

Proteins associated with recovered nanoparticles were quantified by BCA Protein assay kit. Pb values, expressed as μg of protein/μM lipid were then calculated. For the BCA assay, a 6-point standard curve was generated by serial dilutions of BSA in HBS, with the top standard at a concentration of 2 μg/ml. BCA reagent A and B were mixed at a ratio of 50:1 and 200 μl of the BCA mixture were dispensed into a 96-well plate, in duplicates. Then, 25 μl of each standard or unknown sample were added per well. The plate was incubated for 30 minutes at 37° C., after which the absorbance was read at 574 nm on a plate reader (Fluostar Omega). Protein concentrations were calculated according to the standard curve. To quantify lipid concentration, 20 μl of each sample was mixed with 1 ml of chloroform and 500 μl of Stewart assay reagent in an Eppendorf tube. The samples were vortexed for 20 seconds followed by 1 min of centrifugation at 13 000 RPM. 200 μl of the chloroform phase was transferred to a quartz cuvette. The optical density was measured on a using Cary 50 Bio Spectrophotometer (Agilent Technologies) at 485 nm. Lipid concentration was calculated according to a standard curve.

Statistical analysis of the data was performed using IBM SPSS Statistics software. One-way analysis of variance (ANOVA) followed by the Tukey multiple comparison test were used and p values <0.05 were considered significant.

FIG. 11 provides the results for the BCA assay for the human patient study. The level of total amount of protein detected in the patients following in vivo administration is significantly greater than after in vitro incubation of the nanoparticles in plasma taken from such patients.

1. A method of identifying a biomarker from a biofluid, wherein the method comprises:
a. administering a plurality of nanoparticles to a subject in a diseased state to allow a biomolecule corona to form on the surface of said nanoparticles or incubating a plurality of nanoparticles in a biofluid sample taken from a subject in a diseased state to allow a biomolecule corona to form on the surface of said nanoparticles;
b. isolating the nanoparticles and surface-bound biomolecule corona; and
c. analyzing the biomolecule corona to identify the said biomarker.
